(19) United States
(12) Patent Application Publication
(10) Pub. No.: US 2026/0069513 A1
(43) Pub. Date: Mar. 12, 2026

(54) COSMETIC COMPOSITIONS CONTAINING FLATTENED, POROUS CERAMIC PARTICLES AND AT LEAST ONE FILM FORMING AGENT

(71) Applicant: L'OREAL, PARIS (FR)

(72) Inventor: BRADY ZARKET, EDISON, NJ (US)

(73) Assignee: L'OREAL, PARIS (FR)

(21) Appl. No.: 18/826,961

(22) Filed: Sep. 6, 2024

(51) Int. Cl. A61K 8/02; A61Q 1/02; A61Q 19/00

(52) U.S. Cl. CPC A61K 8/0279; A61Q 1/02; A61Q 19/007; A61K 2800/412; A61K 2800/54; A61K 2800/592; A61K 2800/651

(57) ABSTRACT

The invention relates to cosmetic compositions including, in a physiologically acceptable medium, flattened, porous ceramic particles and at least one film forming agent, as well as to methods and kits including such emulsion compositions in container(s) or in application.

### FIELD OF THE INVENTION

The present invention relates to cosmetic compositions comprising, in a physiologically acceptable medium, flattened, porous ceramic particles and at least one film forming agent, as well as to methods and kits comprising such compositions in container(s) or in application. The compositions have beneficial cosmetic properties including, for example, good or improved water and/or sebum absorption, improved mattity, lower modulus properties, and/or good or improved color intensity.

### DISCUSSION OF THE BACKGROUND

At the present time in the market for caring for and making up keratin materials, many products claim staying power throughout the day, withstanding external factors such as water, sebum, mechanical friction, etc. (waterproof mascara, food-proof lipsticks, long-lasting foundations). Unfortunately, many of these compositions do not generally possess good long-wear/transfer-resistance properties as well as good appearance properties (for example, matte properties and/or color intensity) and/or application properties (for example, low viscosity or low modulus).

With respect to lip compositions, commercial products containing silicon resins such as MQ resins are known. Such products are known to provide good long wear properties/transfer-resistance. However, such products possess poor application properties and/or poor feel upon application (for example, feels rough).

Typically, a second composition (topcoat) is separately applied to such products to improve poor properties of the compositions to make the products acceptable to consumers. However, topcoat compositions tend to decrease the long-wear/transfer-resistance properties of the lip compositions, thereby rendering the long-wear/transfer resistant composition less acceptable to consumers and less acceptable for their intended purpose.

Possibly related patent references include:

U.S. Pat. No. 11,806,424, which purports to disclose a cosmetic composition containing (i) at least a plate type filler with refractive index>1.6 and having a particle size between 1 μm and 20 μm, (ii) at least a silicon elastomer, and (iii) at least a filler having an oil absorption capacity greater than or equal to 1 ml/g.

US 2010/0197805, which purports to disclose a paste composition containing at least 10% by weight, relative to its total weight, of filler(s), at least one silicone polyamide block copolymer (PSPA) and at least one wax having a starting melting point of greater than or equal to 50° C.

WO 2018/079455, which purports to disclose at least one plate type filler having a particle size of 10 μm or less, (b) at least one pearlescent pigment having a particle size of 15 μm or less, and (c) at least one hollow or porous particle, wherein the (a) plate type filler has a refractive index of 2.0 or less, preferably 1.9 or less, and more preferably 1.8 or less, and the (b) pearlescent pigment has a colored reflection.

CN106458622, which purports to disclose methods of producing alpha-Al₂O₃ flakes having a D50-value of 5.15 μm, a D80-value of less than 20 μm, a particle thickness of <500 nm and a Mill value (average friction coefficient measured by the KES friction tester) of less than 0.8.

There remains a need for improved cosmetic compositions having improved properties with respect to composition ease of and/or comfort of application of the composition, and/or in particular with respect to improved properties related to improved wear of the cosmetic composition on keratin materials and its resistance to external forces such as mechanical friction, water, sweat and perspiration, sebum and/or oil.

### SUMMARY OF THE INVENTION

The present invention relates to cosmetic compositions comprising, in a physiologically acceptable medium, flattened, porous ceramic particles and at least one film forming agent. Preferably, the composition is a composition for application to skin. Preferably, the composition is a foundation, or a skincare composition such as a moisturizer. Preferably, the composition is in the form of an emulsion. Also preferably, the composition further comprises at least one colorant and/or at least one active agent.

The present invention relates to low storage modulus cosmetic compositions comprising, in a physiologically acceptable medium, flattened, porous ceramic particles and at least one film forming agent. Preferably, the composition is a composition for application to skin. Preferably, the composition is a foundation, or a skincare composition such as a moisturizer. Preferably, the composition is in the form of an emulsion. Also preferably, the composition further comprises at least one colorant and/or at least one active agent.

The present invention also relates to methods of treating, caring for and/or making up keratinous material such as skin by applying compositions of the present invention to the keratinous material in an amount sufficient to treat, care for and/or make up the keratinous material. Preferably, the composition is a composition for application to skin. Preferably, the composition is a foundation, or a skincare composition such as a moisturizer. Preferably, the composition is in the form of an emulsion.

The present invention also relates to methods of increasing matte properties of a cosmetic composition comprising at least one film forming agent, wherein the method comprises adding flattened, porous ceramic particles to the composition during preparation of the composition in an amount sufficient to increase matte properties of the composition.

The present invention also relates to methods of increasing water and/or sebum absorbance properties of a cosmetic composition comprising at least one film forming agent, wherein the method comprises adding flattened, porous ceramic particles to the composition during preparation of the composition in an amount sufficient to increase water and/or sebum absorbance properties of the composition.

The present invention also relates to methods of preparing a lower storage modulus cosmetic composition comprising at least one film forming agent, wherein the method comprises adding flattened, porous ceramic particles to the composition during preparation of the composition in an amount sufficient to prepare a lower storage modulus composition.

### BRIEF DESCRIPTION OF THE FIGURES

FIG. 1 represents a flattened, porous ceramic particle of the present disclosure, including diameter (d) and cross section (c).

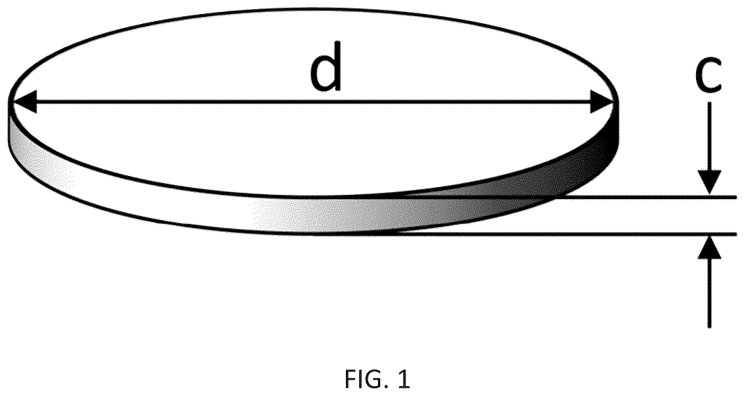

### DETAILED DESCRIPTION OF THE INVENTION

In the following description of the invention and the claims appended hereto, it is to be understood that the terms used have their ordinary and accustomed meanings in the art, unless otherwise specified.

“About” as used herein means within 10% of the indicated number (e.g., “about 10%” means 9%-11% and “about 2%” means 1.8%-2.2%).

“A” or “an” as used herein means “at least one.”

“At least one” means one or more and thus includes individual components as well as mixtures/combinations.

As used herein, all ranges provided are meant to include every specific point and range within, and combination of subranges between, the given ranges. Thus, a range from 1-5, includes specifically 1, 2, 3, 4 and 5, as well as subranges such as and 2-5, 3-5, 2-3, 2-4, 1-4, etc.

“Film former”, “film-forming polymer” or “film-forming agent” as used herein means a polymer or resin that leaves a film on the substrate to which it is applied, for example, after a solvent accompanying the film former has evaporated, absorbed into and/or dissipated on the substrate.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Wax” as used herein is a lipophilic fatty compound that is solid at ambient temperature (25° C.) and changes from the solid to the liquid state reversibly, having a melting temperature of more than 30° C. and, for example, more than 45° C., and a hardness of more than 0.5 MPa at ambient temperature.

“Surfactant” and “emulsifier” are used interchangeably throughout this specification.

“Matte” in compositions as used herein refers to compositions having little to no light reflection. For example, matte compositions can have average gloss properties, measured at 60°, of less than or equal to 10, for example 9, preferably less than or equal to 8, 6, 5, 4 or 1, including all ranges and subranges therebetween such as 1-10, 1-5, 2-10, 3-8, less than 1, less than 3, less than 5, etc. A lower average gloss property value indicates more matteness (or less gloss) for a composition. Such measurements can be made by depositing films to be tested onto a substrate (ex. a black scrub panel P121-10N or opacity card BYK No. 2810) using a drawdown bar (for example, of 1 mil, 3 mil, or 6 mil thickness) and an Automatic Drawdown Machine. The films can then be dried at room temperature and analyzed using a gloss meter (BYK: micro-TRI-gloss) at a desired angle, such as 60° or 85°.

Preferred compositions of the present disclosure are fluid. “Fluid” is understood in particular to mean a composition which is not solid at ambient temperature (25° C.) and for which it is possible to measure a viscosity. The term “fluid” is understood to mean a liquid composition which flows under its own weight at ambient temperature (25° C.) and atmospheric pressure.

Preferably, a fluid composition according to the present disclosure has a Storage modulus (G′) of less than 1100 Pa, preferably less than 600 Pa, preferably less than 400 Pa, preferably less than 300 Pa, and preferably less than 200 Pa. “Modulus” and “storage modulus” are used interchangeably in this specification-“modulus” is used as shorthand for “storage modulus.” Further, in both cases, these terms refer to “shear storage modulus.”

Preferably, the composition of the present disclosure exhibits, at 25° C., a viscosity of between 0.05 and 25 Pa·s and preferably of between 0.1 and 2 Pa·s when measured at a shear rate of about 94 l/s. A suitable method for measuring viscosity is generally carried out at 25° C., using a Rheometer equipped with a 40 mm roughened or sand-blasted stainless steel parallel plate geometry A flow ramp can be performed from 0.01 l/s to 1000 l/s shear rate in log mode with 10 points per decade and viscosity determined from the resulting data.

“Porous” in particles as used herein means particles having void space into which gas or liquids can pass. “Porosity” is a measure of the amount of void space present in a particle, and can be measured with Mercury Intrusion Prosimetry.

“Free” or “substantially free” or “devoid of” as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. Thus, for example, “free of wax” means that an effective amount of wax is omitted from the composition (that is, about 0% by weight), “substantially free of wax” means that wax is present in amounts not greater than 0.25% by weight, and “devoid of wax” means that wax is present in amounts not greater than 0.5% by weight, based on the total weight of the composition. The same nomenclature applies for all other ingredients affirmatively identified throughout the application and in this paragraph such as, for example, (1) surfactants (compositions of the invention which are “free of surfactants,” “substantially free of surfactants,” and “devoid of surfactants,”) and (2) colorants (compositions of the invention which are “free of colorants,” “substantially free of colorants,” and “devoid of colorants”) have meanings consistent with the discussion within this paragraph, even if not specifically discussed in this paragraph. Discussed examples of the use of such language such as those in this paragraph are intended to be exemplary for other ingredients in the application, not limiting.

“Volatile”, as used herein, means having a flash point of less than about 100° C.

Non-volatile”, as used herein, means having a flash point of greater than about 100° C.

“Polymer” as used herein means a compound which is made up of at least two monomers.

“Anhydrous” as it is used herein means that compositions of the invention contain less than 3% water, meaning that the compositions can also contain less than 2% water, and less than 1% water, as well as being “free of water,” “substantially free of water,” and “devoid of water” as defined above.

“Keratinous materials” means nails (fingernails and/or toenails), skin such as body, face, and eye area, scalp, keratin fibers such as eyelashes, eyebrows, and hair, and mucous membranes such as lips.

“Physiologically acceptable medium” means a medium or carrier which is compatible with keratinous materials, having a pleasant color, odor and feel, and not causing any unacceptable discomfort (stinging or tautness) liable to discourage a consumer from using the composition.

“Makeup Result” as used herein, refers to compositions where makeup properties such as color, gloss, matte or transfer-resistance remains the same or substantially the same as at the time of application, as viewed by the naked eye of one of ordinary skill in the art, after contact such as rubbing and/or after an extended period of time. “Makeup Result” may be evaluated by evaluating makeup properties by any method known in the art for evaluating such properties. For example, long wear or transfer-resistance may be evaluated by a test involving the application of a composition to keratinous material such as skin and evaluating the matte properties of the composition after rubbing with a substrate and/or an extended period of time. For example, the matte properties of a composition may be evaluated immediately following application to keratinous material such as skin and these characteristics may then be re-evaluated and compared. Further, these characteristics may be evaluated with respect to other compositions, such as commercially available compositions.

“Natural” as in the phrase “natural compound” refers to any compound derived directly from a natural substance such as a plant without having undergone any chemical modification.

“Compound of natural origin” refers to any compound derived from a natural compound which has undergone one or more chemical modifications, for example by organic synthesis reaction, without the properties of the natural compound having been modified.

“Synthetic compound” refers to any compound which is not a natural compound or a compound of natural origin.

“Room temperature” means 25° C.

“Atmospheric pressure” means 760 mmHg, i.e., 105 pascals.

The compositions and methods of the present invention can comprise, consist of, or consist essentially of the essential elements and limitations of the invention described herein, as well as any additional or optional ingredients, components, or limitations described herein or otherwise useful. For example, the oil and/or water absorbing component the compositions of the invention can “consist essentially of” flattened, porous ceramic particles.

For purposes of the present invention, the “basic and novel property” associated with compositions, components and methods which “consist essentially of” identified ingredients or actions is “reduced storage modulus (G′).” However, when the compositions or methods of the present invention also comprise colorants in an amount effective to provide color to keratinous materials to which the compositions or methods have been applied, the “basic and novel property” associated with compositions, components and methods which “consist essentially of” identified ingredients or actions can include “color intensity enhancement.”

Referred to herein are trade names for materials including, but not limited to, materials such as polymers and optional components. Materials are not intended to be limited by materials described and referenced by a certain trade name herein. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Composition

### Flattened, Porous Ceramic Particles

In accordance with the present invention, compositions comprising flattened, porous ceramic particles are provided. Preferably, the flattened, porous ceramic particles comprise at least one metal oxide, with alumina being the most preferred metal oxide. Suitable examples of acceptable flattened, porous ceramic particles include those alumina particles available from Eurekite under the name Flexiramics®, and examples of such particles are described in patent application(s) filed on the same date as the present application by the supplier.

Preferably, the flattened, porous ceramic particles have a porosity of from about 5% to about 60%, preferably from about 10% to about 50%, preferably from about 15% to about 50%, and preferably from about 15% to about 45%, as measured by Mercury intrusion porosimetry (MIP), including all ranges and subranges therein. Preferably, porosity of the flattened, porous ceramic particles is sufficient to inhibit visible light reflection by the ceramic particles such that matte properties of a composition containing the flattened, porous ceramic particles may be improved, preferably by at least about 10% as compared to non-porous (less than about 5% porosity) ceramic particles, preferably by at least about 20% as compared to non-porous ceramic particles.

Preferably, the pores in the flattened, porous ceramic particles have of a size of from about 50 nm to about 1000 nm, preferably with an average size of from about 100 nm to about 500 nm, as measured by Mercury intrusion porosimetry (MIP), including all ranges and subranges therebetween.

Preferably, the flattened, porous ceramic particles have of a flattening property (f) of at least about 0.3, preferably at least about 0.5, preferably at least about 0.6, preferably at least about 0.7, preferably at least about 0.8, and preferably at least about 0.9, where flattening (f) is determined as follows: the flattened, porous ceramic particles generally have half-spherical character (platelet-like shape) having associated with it a diameter (d) and a cross section (c). Flattening (f) can be defined:

$$f = ( d - c ) / d .$$

Preferably, the diameter (d) of the flattened, porous ceramic particles is greater than 15 microns (μm), preferably greater than 20 microns, preferably greater than 25 microns, and preferably greater than 50 microns, such as, for example, 20 microns to 200 microns, 30 microns to 175 microns, 25 microns to 500 microns, etc., including all ranges and subranges therebetween.

“Flattened” as used herein means a particle in which diameter (d) is at least twice as large as cross section (c).

According to preferred embodiments, the flattened, porous ceramic particles are present in the compositions of the present invention in an amount ranging from about 0.5% to about 20% by weight, preferably from about 1 to about 15% by weight, preferably from about 1.5 to about 10% by weight, and preferably from about 2% to about 7.5% by weight, based on the total weight of the composition, including all ranges and subranges within these ranges such as, for example, 1.25% to 8%, 1.4% to 7%, 2% to 6%, etc. Preferably, the flattened, porous ceramic particles are present in the color compositions of the present disclosure in an amount sufficient to increase color intensity of the composition as compared to the same composition but lacking the flattened, porous ceramic particles.

### Film Forming Agent

According to the present invention, compositions comprising at least one film forming agent are provided. The film forming agent(s) useful in compositions of the present disclosure have a hydrocarbon-based backbone and silicone substitution(s) or sidechain(s).

Suitable examples of hydrocarbon-based backbones for film forming agents of the present invention include, but are not limited to, polysaccharides, high viscosity esters, polybutenes, polyisobutenes, polyhydrogenated butenes, acrylic polymers, acrylate copolymers, vinyl pyrrolidone (VP) containing homopolymers and copolymers, polyurethanes, polyolefins, silicone acrylate copolymers, and mixtures thereof.

Suitable examples of hydrocarbon-based backbones include, but are not limited to, homopolymers and copolymers containing at least one (meth)acrylic acid ((meth)acrylate) monomer and/or at least one vinylpyrrolidone monomer such as polyvinylpyrrolidone, vinyl pyrrolidone/vinyl acetate copolymers, vinyl pyrrolidone/acrylic acid copolymers, vinyl pyrrolidone/acrylate copolymers, alkyl (e.g., butyl) acrylate/hydroxyalkyl (e.g., propyl) acrylate copolymers, styrene/acrylates/ammonium methacrylate copolymers, acrylamide/sodium acryloyldimethyltaurate copolymer, etc.; water-soluble polysaccharides such as pectin, unmodified pullulan or cellulose compounds; polyurethanes; latexes; and mixtures thereof.

Suitable examples of silicone substitution(s) or sidechain(s) include trialkylsiloxysilicates such as trimethylsiloxysilicates, organopolysiloxanes such as, for example, non-elastomeric organopolysiloxanes including polydimethylsiloxanes such as dimethicone, polymethylhydrogenosiloxanes and polyalkoxydimethylsiloxanes; silane derivatives such as, for example, alkoxysilanes such as the alkyltriethoxysilanes and the alkyltrimethoxysilanes.

Suitable examples of specific hydrocarbon-based backbone film forming agents having silicone substitution(s) or sidechain(s) include, but are not limited to, silicone-modified pullulan compounds such as trialkylsiloxysilylcarbamoyl pullulans, with a particularly preferred compound being trimethylsiloxysilylcarbamoyl pullulan; non-polar silicone copolymers comprising repeating units of at least one polar (meth)acrylate unit and vinyl copolymers grafted with at least one non-polar silicone chain. Non-limiting examples of non-dendrimer copolymers are acrylates/dimethicone copolymers such as those commercially available from Shin-Etsu, for example, the products sold under the tradenames KP-545 (cyclopentasiloxane (and) acrylates/dimethicone copolymer), KP-543 (butyl acetate (and) acrylates/dimethicone copolymer), KP-549 (methyl trimethicone (and) acrylates/dimethicone copolymer), KP-550 (INCI name: isododecane (and) acrylate/dimethicone copolymer), KP-561 (acrylates/stearyl acrylate/dimethicone acrylates copolymer), KP-562 (acrylates/behenyl acrylate/dimethicone acrylates copolymer), and mixtures thereof. Additional examples include the acrylate/dimethicone dendrimer copolymers sold by Dow Corning under the tradenames FA 4001 CM SILICONE ACRYLATE (cyclopentasiloxane (and) acrylates/polytrimethylsiloxymethacrylate copolymer) and FA 4002 ID SILICONE ACRYLATE (isododecane (and) acrylates/polytrimethylsiloxymethacrylate Copolymer), and mixtures thereof.

Preferably, the at least one film forming agent is/are present in the compositions of the present invention in an amount ranging from about 0.5% to about 25% by weight, preferably from about 1% to about 20% by weight, preferably from about 2.5% to about 17.5%, and preferably from about 5% to about 15% by weight with respect to the weight of the composition, including all ranges and subranges therebetween such as, for example, 4% to 14%, 1% to 15%, 5% to 25%, etc.

### Oil

According to preferred embodiments of the present invention, compositions further comprising at least one oil are provided. “Oil” means a fatty substance which is liquid at room temperature (20 to 25° C.) and atmospheric pressure (760 mm Hg). However, compositions of the present invention may be “free of,” “substantially free of,” or “devoid of” oil as defined above.

Suitable include volatile and/or non-volatile oils. Such oils can be any acceptable oil including but not limited to silicone oils and/or hydrocarbon oils.

According to certain embodiments, the compositions of the present invention preferably comprise one or more volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |

Further, a volatile linear silicone oil may be employed in the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to certain embodiments of the present invention, the composition of preferably comprises one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isohexacecane, isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 2 below.

TABLE 2

| | Flash Point |
|---|---|
| Compound | (° C.) |
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

The volatility of the solvents/oils can be determined using the evaporation speed as set forth in U.S. Pat. No. 6,338,839, the contents of which are incorporated by reference herein.

According to certain embodiments of the present invention, the composition comprises at least one non-volatile oil. Examples of non-volatile oils that may be used in the present invention include, but are not limited to, polar oils such as, for example:
caprylic/capric acid triglycerides, for instance those sold by the company Stearineries Dubois or those sold under the names Miglyol 810, 812 and 818 by the company Dynamit Nobel;
esters and synthetic ethers, in particular fatty acids, such as oils of formulas RICOOR2, wherein R 1 represents the remainder of a fatty acid having from 8 to 29 carbon atoms, and R 2 represents a hydrocarbon chain, branched or not, containing from 3 to 30 carbon atoms, such as for example Purcellin oil, isononyl isononanoate, isopropyl myristate, ethyl-2-hexyl palmitate, octyl-2-dodecyl stearate, octyl-2-dodecyl erucate, isostearyl isostearate; hydroxylated esters such as isostearyl lactate, octylhydroxystearate, octyldodecyl hydroxystearate, diisostearylmalate, triisocetyl citrate, heptanoates, octanoates, fatty alcohol decanoates; polyol esters, such as propylene glycol dioctanoate, neopentylglycol diheptanoate and diethylene glycol diisononanoate; and esters of pentaerythritol such as pentaerythrityl tetraisostearate or dipentaerythrityl pentaisononanoate;
ethers containing from 10 to 40 carbon atoms;
liquid C₈ to C₂₆ fatty alcohols, for instance oleyl alcohol, cetyl alcohol, stearyl alcohol, octyldodecanol, and cetearly alcohol;
hydrocarbon oils of animal origin, such as perhydrosqualene;
hydrocarbon oils of vegetable origin, such as liquid triglycerides of fatty acids having from 4 to 10 carbon atoms such as triglycerides of heptanoic or octanoic acids or, for example, sunflower, corn, soybean, squash, grape seed, sesame, hazelnut, apricot, macadamia, ara, sunflower oil, castor oil, avocado, triglycerides of caprylic/capric acids such as those sold by the company Stearineries Dubois or those sold under the names Miglyol 810, 812 and 818 by the company Dynamit Nobel, coco-caprylate/caprate (esterified oil from coconut oil), jojoba oil, shea butter oil; and
mixtures thereof.

Further, examples of non-volatile oils that may be used in the present invention include, but are not limited to, non-polar oils such as branched and unbranched hydrocarbons, in particular Vaseline (petrolatum), paraffin oil, squalane, squalene, hydrogenated polyisobutene, hydrogenated polydecene, polybutene, mineral oil, pentahydrosqualene, and mixtures thereof.

According to certain embodiments of the present invention, the compositions of the present invention comprise at least one non-volatile silicone oil. Suitable examples of such silicone oils include, but are not limited to, non-volatile silicone fluids such as, for example, polyalkyl (aryl) siloxanes. Suitable polyalkyl siloxanes include, but are not limited to, polydimethyl siloxanes, which have the CTFA designation dimethicone, polydiethyl siloxane, phenyl trimethicone, trimethyl pentaphenyl trisiloxane, phenyldimethicone, phenyltrimethylsiloxydiphenylsiloxane, diphenyldimethicone, and diphenylmethyldiphenyltrisiloxane and those siloxanes disclosed in U.S. patent application publication no. 2004/0126350, the entire disclosure of which is hereby incorporated by reference. Specific examples of suitable high viscosity silicone oils include, but are not limited to, 15 M 30 from PCR (500 cSt) or Belsil PDM 1000 (1000 cSt) from Wacker and Dow Corning 200 (350 cSt) (the values in parenthesis represent viscosities at 25° C.).

Particularly preferred oils include, but are not limited to, one or more of the following: Diisopropyl Sebacate, C12-15 Alkyl Benzoate, Phenethyl Benzoate, Isopropyl Lauroyl Sarcosinate, Diisopropyl Adipate, Dibutyl Adipate, Dicaprylyl Carbonate, Dicaprylate/Dicaprate, Coco Glycerides, Caprylic/capric triglyceride, Isopropyl Myristat, Isopropyl Palmitate, Coco Caprylate/Caprate, Ethylhexyl Palmitate, Isononyl isononanoate, Octyl dodecanol, Isohexadecane, isododecane, Dicaprylyl Ether, C15-19 Alkane, and mixtures thereof.

According to preferred embodiments, the at least one oil is/are present in the compositions of the present invention in an amount ranging from about 1% to about 50% by weight, more preferably from about 5 to about 40% by weight, and preferably from about 10% to about 35% by weight, based on the total weight of the composition, including all ranges and subranges within these ranges such as, for example, 15% to 40%, 20% to 45%, etc.

### Aqueous Phase

The compositions of the present invention may also contain water, if desired. When the compositions of the present invention contain water, they are preferably in the form of an emulsion, and further comprise at least one surfactant. Preferably, when the compositions of the present invention contain water, they are in the form of an emulsion containing an external aqueous phase such as an oil-in-water emulsion (O/W) or a water-in-oil-in-water emulsion (O/W/O), or an emulsion containing an external oil phase such as a water-in-oil emulsion (W/O) or an oil-in-water-in-oil (O/W/O) emulsion. Preferably, when in the form of an emulsion, the oil phase can contain silicone oils (e.g., Si/W or W/Si emulsion) or hydrocarbon oils.

When present, water is preferably present in an amount of from about 10% to about 80% by weight, preferably from about 20% to about 70% by weight, preferably from about 35% to about 65% by weight, including all ranges and subranges therebetween, all weights being based on the total weight of the composition.

According to preferred embodiments, however, compositions of the present invention are devoid of water, substantially free of water, or free of water as defined herein. Preferably, compositions of the present invention are anhydrous.

If present in compositions of the present invention, the aqueous phase may further comprise at least one water-soluble organic solvent which is liquid at room temperature and atmospheric pressure. For example, such at least one water-soluble organic solvent may include:
C1-C5 monoalcohols having a C1-C5 alkane chain and a single hydroxyl function (OH). Suitable C1-C5 monoalcohols include methanol, ethanol, propanol, isopropanol, butanol and mixtures thereof;
polyols (compounds having 2 or more hydroxyl groups) having, for example from 2 to 20 carbon atoms, preferably from 2 to 6 carbon atoms, such as for example glycerol, diglycerol, propylene glycol, isoprene glycol, dipropylene glycol, butylene glycol, hexylene glycol, 1,3-propanediol, pentylene glycol, simple sugars, and water-soluble polyalkylene glycols;
esters of short-chain (C2-C5) carboxyl-containing compounds such as citrates, such as for example triethyl citrate;
and mixtures thereof.

According to preferred embodiments, the at least one water-soluble organic solvent is selected from the group consisting of ethanol, triethyl citrate, dipropylene glycol, butylene glycol, propanediol and propylene glycol, and mixtures thereof.

If present, the at least one water-soluble organic solvent is preferably present in compositions of the present invention in an amount ranging from about 0.5 to about 40% by weight, preferably from about 3 to about 30% by weight, and preferably from about 5% to about 20% by weight relative to the total weight of the composition, including all ranges and subranges therebetween such as, for example, 2% to 15%, 2% to 25%, 7.5% to 30%, etc.

### Coloring Agent

According to preferred embodiments the present invention, compositions further comprising at least one coloring agent are provided. Preferably, the at least one coloring agent is chosen from the group consisting of soluble dyes, pigments, nacres and glitter.

The term “soluble dyes” should be understood to refer to organic, inorganic or organometallic compounds, soluble in the composition according to the invention and intended to color said composition.

Suitable dyes are, for example, Sudan Red, DC Red 17, DC Green 6, β-carotene, soybean oil, Sudan brown. DC Yellow 11, DC Violet 2, DC Orange 5 and Quinoline Yellow.

The term “nacres” should be understood to refer to iridescent particles of any shape, particularly produced by some mollusks in their shell or by synthetic means.

The term “pigments” should be understood to refer to inorganic or organic, white or colored particles of any shape, insoluble in the composition according to the invention and intended to color said composition.

The pigments may be white or colored, inorganic and/or organic. Of the inorganic pigments, mention may be made of titanium dioxide, optionally surface-treated, zirconium or cerium oxides, along with zinc, iron (black, yellow or red) or chromium oxides, manganese violet, ultramarine blue, chromium hydrate and iron blue, metallic powders such as aluminum powder, copper powder.

Of the organic pigments, mention may be made of carbon black, D & C type pigments, and lacquers based on cochineal carmine, barium, strontium, calcium, aluminum.

Mention may also be made of effect pigments such as particles comprising a natural or synthetic organic or inorganic substrate, for example glass, acrylic resins, polyester, polyurethane, polyethylene terephthalate, ceramics, aluminas and optionally coated with metallic substances such as aluminum, gold, copper, bronze, or with metal oxides such as titanium dioxide, iron oxide, chromium oxide, inorganic or organic pigments and mixtures thereof.

The pearlescent pigments may be chosen, for example, from pigments based on silica, titanium dioxide and mica, including for example mica-based pigments sold under the tradename Spectrval by Merck KGaA, white pearlescent pigments such as mica coated with titanium, or bismuth oxychloride, colored pearlescent pigments such as titanium mica coated with iron oxides, titanium mica coated with iron blue and chromium oxide in particular, titanium mica coated with an organic pigments of the aforementioned type and pearlescent pigments based on bismuth oxychloride.

Pigments with goniochromatic properties may be used, particularly liquid crystal or multilayer pigments.

Optical brighteners or fibers optionally coated with optical brighteners may also be used.

If present, the at least one coloring agent is present in the compositions of the present invention in an amount effective to provide visible color to keratinous material after application of the composition to the keratinous material, preferably ranging from about 0.1% to about 50% by weight, preferably from about 0.2% to about 40% by weight, preferably from about 0.5% to about 25%, preferably from about 1% to about 10%, and preferably from about 1% to about 5% by weight with respect to the weight of the composition, including all ranges and subranges therebetween such as, for example, 2% to 15%. 25% to 50%, 3% to 8%, etc. However, the compositions of the present disclosure can be free of, substantially free of, or devoid of coloring agent as defined above.

### Wax

According to preferred embodiments the present invention, compositions further comprising at least one wax are provided. Preferably, the at least one wax has a melting point of at least 40° C., preferably at least 50° C., and preferably at least 60° C.

Suitable examples of waxes that can be included in compositions of the present invention include those generally used in the cosmetics field such as, for example, synthetic waxes, silicone waxes, fatty alcohol waxes, waxes of natural origin, etc.

Specific waxes of natural origin include, in particular, beeswax and vegetable waxes, and include waxes such as carnauba wax, candelilla wax, ouricoury wax, Japan wax, sunflower seed wax, cork fibre wax or sugar cane wax, (rice) bran wax, montan wax, paraffin wax, lignite wax or microcrystalline wax, ceresin or ozokerite, with beeswax, carnauba wax and (rice) bran wax being preferred.

Examples of silicone waxes, if present, include, but are not limited to, silicone waxes such as alkyl- or alkoxydimethicones having an alkyl or alkoxy chain ranging from 10 to 45 carbon atoms, poly(di)methylsiloxane esters which are solid at 30° C. and whose ester chain comprising at least 10 carbon atoms, di(1,1,1-trimethylolpropane)tetrastearate, which is sold or manufactured by Heterene under the name HEST 2T-4S; alkylated silicone acrylate copolymer waxes comprising at least 40 mole % of siloxy units having the formula (R₂R′SiO_{1/2})ₓ(R″SiO_{3/2})_{y}, where x and y have a value of 0.05 to 0.95, R is an alkyl group having from 1 to 8 carbon atoms, an aryl group, a carbinol group, or an amino group, R is a monovalent hydrocarbon having 9-40 carbon atoms, R″ is a monovalent hydrocarbon group having 1 to 8 carbon atoms, an aryl group such as those disclosed in U.S. patent application 2007/0149703, the entire contents of which is hereby incorporated by reference, with a particular example being C30-C45 alkyldimethylsilyl polypropylsilsesquioxane; and mixtures thereof.

Fatty alcohol waxes preferably contain from about 8 to about 30 carbon atoms, preferably from about 12 to about 28 carbon atoms, and preferably from about 14 to about 24 carbon atoms, including but not limited to 8 to 24 carbon atoms, 12 to 16 carbon atoms, 16 to 22 carbon atoms, etc. The fatty alcohol wax can be saturated or unsaturated, linear or branched. Mention may be made, for example, of cetyl alcohol, stearyl alcohol, behenyl alcohol, and mixtures thereof. Other examples of suitable fatty alcohol waxes include waxes including natural components (such as from one or more natural oils) and fatty alcohol components such as, for example, hydrogenated stearyl olive ester, commercially available from the supplier Sophim under the tradename Phytowax Olive 18 L 57, and hydrogenated myristyl olive ester, commercially available from the supplier Sophim under the tradename Phytowax Olive 14 L 48.

If present, the at least one wax is/are preferably present in an amount of from about 0.5% to about 30% by weight, preferably from about 1% to about 25% by weight, preferably from about 2.5% to about 25% by weight, and preferably from about 5% to about 20% by weight, including all ranges and subranges therebetween such as, for example, from about 1% to about 12% by weight, from about 2% to about 10% by weight, from about 15% to about 25% by weight, and from about 2% to about 8% by weight, with all weights being based on the total weight of the composition.

According to certain embodiments, the composition contains less than 10% by weight of beeswax, preferably less than 5% by weight of beeswax, preferably less than 2.5% by weight of beeswax, or preferably is “substantially free,” “devoid” of or “free” of beeswax as defined above.

According to certain embodiments, the composition contains less than 10% by weight of synthetic wax, preferably less than 5% by weight of synthetic wax, preferably less than 2.5% by weight of synthetic wax, or preferably is “substantially free,” “devoid” of or “free” of synthetic wax as defined above.

According to certain embodiments, the composition contains less than 10% by weight of silicone wax, preferably less than 5% by weight of silicone wax, preferably less than 2.5% by weight of silicone wax, or preferably is “substantially free,” “devoid” of or “free” of silicone wax as defined above.

### Thickening Agent

According to preferred embodiments of the present invention, compositions further comprising at least one thickening agent are provided. Suitable thickening agents include agents for thickening an oil phase of a composition, agents for thickening a water phase of a composition, and mixtures thereof. However, it is to be understood that preferred embodiments of the present invention include compositions which are “free,” “substantially free” or “devoid” of thickening agents (compositions are “free of thickening agents,” “substantially free of thickening agents,” or “devoid of thickening agents”), particularly when the compositions are low viscosity compositions.

According to preferred embodiments, the thickening agent is a gelling agent for gelling water (water phase) in the composition. Suitable gelling agents include any gelling agent, such as for example, clays and acrylic acid (co) polymers, such as high molecular weight homo- or co-polymers comprising acrylic acid, optionally crosslinked with a polyalkenyl polyether, including some polymers identified as “carbomer” as well as amphiphilic polymers. Preferably, the gelling agent is an amphiphilic polymer or a clay, or a combination thereof.

According to preferred embodiments, the gelling agent(s) may be at least one clay. Among the clays, mention may be made of clays of the smectite family, such as laponite, of the kaolinite family, such as kaolinite, dickite, nacrite, optionally modified clays of the halloysite, dombassite, antigorite, berthierine, pyrophyllite, montmorillonite, beidellite, vermiculite, talc, stevensite, hectorite, saponite, chlorite, sepiolite and illite family.

Clays are products that are already well known per se, which are described, for example, in the publication “Mineralogie des argiles” [“Clay Mineralogy”], S. Caillère, S. Hénin, M. Rautureau, 2nd Edition 1982, Masson, the teaching of which is included herein by way of reference.

Natural clay is a sedimentary rock composed to a large extent of specific minerals, silicates generally of aluminium. Kaolin is thus a natural clay.

The clays may also be synthetic. Thus, Sumecton mentioned below is a synthetic saponite.

The clays may also be chemically modified with various compounds, such as acrylic acids, polysaccharides (for example carboxymethylcellulose) or organic cations.

Preferably, in the context of the present invention, use is made of clays that are cosmetically compatible and acceptable with keratin materials. Clays that may especially be mentioned include kaolinite, montmorillonites, saponites, laponites, hectorites, and illites. Mixtures of clays and natural clays may also be used.

Natural clays that may be mentioned include green clays, in particular rich in illite; clays rich in montmorillonite, known as fuller's earth, or such as bentonite or else white clays rich in kaolinite. As bentonites, or modified bentonites, mention may in particular be made of those sold under the names Bentone 38 VCG®, Bentone Gel CAO V®, Bentone 27 V®, Bentone Gel ISD V, and Bentone Gel MIO VR by the company Elementis.

By way of clay rich in montmorillonite, mention may be made of the aluminium silicate hydrate sold under the name Gel White HR by the company Rockwood.

By way of saponite, which belongs to the montmorillonite family, mention may be made of synthetic saponite, in particular the product sold by the company Kunimine under the name Sumecton®.

By way of tack, mention may be made of those sold under the names Rose Talc® and Talc SG-2000® sold by the company Nippon Talc, Luzenac Pharma M® sold by the company Luzenac, J-68BC® from US Corporation and Micro ACE-P-3® sold by the company Nippon Talc.

Preferably, if present, the thickening agent(s) is/are present in the compositions of the present invention in amounts ranging from about 0.1 to about 30% by weight, preferably from 0.5 to 25% by weight, preferably from 1 to 20% and preferably from 2.5 to 15% by weight, all weights based on the weight of the composition as a whole, including all ranges and subranges therebetween such as, for example, 0.1 to 1.5%, 2 to 20%, 10 to 20%, etc.

### Active Agent

According to preferred embodiments of the present invention, compositions further comprising at least one active agent are provided. Preferably, the active agent is in the aqueous phase (hydrophilic active agent), although active agents may be present in the oil phase (hydrophobic active agent).

In some embodiments, the active agent may be:
a moisturizing agent, such as a polyol such as, for example, glycerin and sugars, urea, and its derivatives, such as in particular hydroxyalkyl urea, in particular hydroxyalkylurea, and mixtures thereof;
a desquamating agent, which may be a compound capable of acting either directly on desquamation by promoting exfoliation, such as β-hydroxy acids, in particular salicylic acid and its derivatives (including 5-n-octanoylsalicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol and certain jasmonic acid derivatives; or acting on the enzymes involved in the desquamation or degradation of corneodesmosomes, glycosidases, stratum corneum chymotryptic enzyme (SCCE), or even other proteases (trypsin, chymotrypsin-like). Mention may be made of agents for chelating mineral salts: EDTA; N-acyl-N,N′,N′-ethylenediaminetriacetic acid; aminosulfonic compounds and in particular (N-2-hydroxyethylpiperazine-N-2-ethane) sulfonic acid (HEPES): 2-oxothiazolidine-4-carboxylic acid (procysteine) derivatives; α-amino acid derivatives of the type such as glycine (as described in EP-0 852 949 and sodium methylglycinediacetate sold by BASF under the trade name Trilon M); boney; sugar derivatives such as O-octanoyl-6-D-maltose and N-acetylglucosamine;
a humectant;
an anti-aging agent, which may include, for example, one or more of C-beta-D-xylopyranoside-2-hydroxypropane (Pro-Xylane), retinol, peptides, caffeine, and other components that provide improvement to skin texture, any other suitable soluble/dispersible targeted active ingredient, and combinations thereof;
a mattifying agent, which may include, but is not limited to, mattifying fillers such as, for example, talc, silica, coated silicas such as silica silylate, perlite, silicone elastomers, and polyamides, and waxes such as, for example, beeswax and copernicia cerifera (carnauba) wax;
an antimicrobial agent, non-limiting examples of which include 2,4,4′-trichloro-2′-hydroxydiphenyl ether (or triclosan), 3,4,4′-trichlorobanilide, phenoxyethanol, phenoxypropanol, phenoxyisopropanol, hexamidine isethionate, metronidazole and its salts, micronazole and its salts, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, 2,4,4′-trichloro-2′-hydroxydiphenyl ether, 3,4,4′-trichlorocarbanalide, octopirox, octoxyglycerine, octanoylglycine, caprylyl glycol. 10-hydroxy-2-decanoic acid, dichlorophenylimidazole dioxolane and its derivatives described in patent WO 93/18743, farnesol and phytosphingosines, and mixtures thereof;
a pigment modifying agent and/or skin lightening agents, such as double-stranded RNA oligonucleotides are useful for decreasing tyrosinase expression. Mention may also be made of ceramides, vitamin C and derivatives thereof, in particular vitamin CG, CP and 3-0 ethyl vitamin C, alpha- and beta-arbutin, ferulic acid, kojic acid, resorcinol and derivatives thereof, calcium D-pantetheine sulphonate, lipoic acid, ellagic acid, vitamin B3, phenylethyl resorcinol, for instance Symwhite 377® from the company Symrise, a kiwi fruit (Actinidia chinensis) juice sold by Gattefosse, an extract of Paeonia suffructicosa root, such as the product sold by the company Ichimaru Pharcos under the name Botanpi Liquid B®, an extract of brown sugar (Saccharum officinarum), such as the extract of molasses sold by the company Taiyo Kagaku under the name Molasses Liquid, a mixture of undecylenic acid and undecylenoyl phenyl alanine, such as Sepiwhite MSH® from Seppic;
an amino acid such as arginine, histidine, methionine, lysine, proline, leucine, lycine, sarcosine, etc.;
a vitamin such as a vitamin A compound such as retinol or retinoic acid, vitamin B compound such as vitamin B1, B3 or B6, in particular niacinamide, a vitamin C compound such as an ascrobyl phosphate salt (e.g., magnesium or sodium, and/or derivatives thereof;
an ultraviolet (UV) filter such as, e.g., an aminobenzoic acid derivative, a dibenzoylmethane derivative, a salicylic acid derivative, a cinnamic derivative, a β,β diphenylacrylate derivative, a benzophenone derivative, benzylidene camphor derivative, and mixtures thereof. Mention may be made especially of ethylhexyl methoxycinnamate sold under the tradename UVINUL MC 80® by the company BASF, of ethylhexyl salicylate sold under the tradename NEO HELIOPAN OSO by the company SYMRISE and of octocrylene sold under the tradename NEO HELIOPAN 303® by the company SYMRISE; and/or a combination thereof.

### Additional Additives

The composition of the invention can also comprise any additive usually used in the field under consideration. For example, dispersants such as poly(12-hydroxystearic acid), preserving agents, fragrances, fillers (preferably fillers capable of absorbing water or sweat), antioxidants, neutralizing agents, silicone elastomers, and mixtures thereof can be added. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9^{th} ed. 2002). However, it is to be understood that preferred embodiments of the present invention include compositions which are “free,” substantially free” or “devoid” of the ingredients discussed in this paragraph such as fillers, in particular mattifying fillers (compositions are “free of mattifying fillers,” “substantially free of mattifying fillers,” or “devoid of mattifying fillers”).

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

Needless to say, the composition of the invention should be cosmetically or dermatologically acceptable, i.e., it should contain a non-toxic physiologically acceptable medium and should be able to be applied to the skin of human beings.

The compositions according to the invention may be in all the galenical forms conventionally used for a topical application and especially in the form of aqueous or aqueous-alcoholic solutions, of oil-in-water (O/W), water-in-oil (W/O) or multiple (triple: W/O/W or O/W/O) emulsions, of aqueous gels or of dispersions of a fatty phase in an aqueous phase using spherules, it being possible for these spherules to be lipid vesicles of ionic and/or non-ionic type (liposomes, niosomes or oleosomes). These compositions are prepared according to the usual methods.

Advantageously, the compositions according to the invention are in the form of a gel, of an emulsion, of a powder or of a paste.

In addition, the composition according to the invention may be more or less fluid and may have the appearance of a white or coloured cream, an ointment, a milk, a lotion, a serum, a paste, a foaming gel, a care product, a tonic or a foam. It may optionally be applied to the skin in aerosol form. It may also be in solid form, and for example in the form of a stick. According to other preferred embodiments, methods of treating, caring for and/or enhancing the appearance of keratinous material by applying compositions of the present invention to the keratinous material in an amount sufficient to treat, care for and/or enhance the appearance of the keratinous material are provided. In accordance with these preceding preferred embodiments, the compositions of the present invention are applied topically to the desired area of the keratinous in an amount sufficient to treat, care for and/or enhance the appearance of the keratinous material. The compositions may be applied to the desired area as needed, preferably once or twice daily, more preferably once daily and then preferably allowed to dry before subjecting to contact such as with clothing or other objects (for example, a topcoat). Preferably, the composition is allowed to dry for about 1 minute or less, more preferably for about 45 seconds or less. The composition is preferably applied to the desired area that is dry or has been dried prior to application, or to which a basecoat has been previously applied.

According to other preferred embodiments, methods of increasing matte properties of a cosmetic composition comprising at least one film forming agent, wherein the method comprises adding flattened, porous ceramic particles to the composition during preparation of the composition in an amount sufficient to increase matte properties of the composition are provided.

According to other preferred embodiments, methods of increasing water and/or sebum absorbance properties of a cosmetic composition comprising at least one film forming agent, wherein the method comprises adding flattened, porous ceramic particles to the composition during preparation of the composition in an amount sufficient to increase water and/or sebum absorbance properties of the composition are provided.

According to other preferred embodiments, methods of preparing a low storage modulus cosmetic composition comprising at least one film forming agent, wherein the method comprises adding flattened, porous ceramic particles to the composition during preparation of the composition in an amount sufficient to prepare a low storage modulus composition are provided.

According to preferred embodiments of the present invention, kits comprising, as separate compositions in one or more containers within the kits, (A) at least one composition of the present invention as discussed above, and (B) at least one other composition such as a primer coat composition, a basecoat composition, or a topcoat composition are provided. Compositions (A) and (B) may be contained in different portions or sections of the same container within the kit. However, compositions (A) and (B) may also be in different containers with the kit.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example 1—Composition Preparation

The following compositions were prepared by making the water phase and the oil phase separately, and then combining the phases. The pigments and filler particles were added to the oil phase after preparing the unpigmented base. After combining the phases, fillers and pigments the compositions were speedmixed until homogeneous.

| | Invention A | Comparative B | Comparative C | Comparative D |
|---|---|---|---|---|
| | (Porous Alumina) | (Perlite) | (P Silica Silylate) | (No filler) |
| INCI US NAME | | | | |
| WATER | 32.02 | 32.02 | 32.02 | 32.02 |
| BUTYLENE GLYCOL | 2.61 | 2.61 | 2.61 | 2.61 |
| MAGNESIUM SULFATE | 0.70 | 0.70 | 0.70 | 0.70 |
| CELLULOSE GUM | 0.09 | 0.09 | 0.09 | 0.09 |
| PHENOXYETHANOL | 0.26 | 0.26 | 0.26 | 0.26 |
| ALCOHOL DENAT. | 4.35 | 4.35 | 4.35 | 4.35 |
| POLYGLYCERYL-4 | 2.61 | 2.61 | 2.61 | 2.61 |
| DIISOSTEARATE/ | | | | |
| POLYHYDROXY- | | | | |
| STEARATE/ | | | | |
| SEBACATE | | | | |
| ISOHEXADECANE | 8.70 | 8.70 | 8.70 | 8.70 |
| ISODODECANE | 12.18 | 12.18 | 12.18 | 12.18 |
| DISTEARDIMONIUM | 13.05 | 13.05 | 13.05 | 13.05 |
| HECTORITE (and) | | | | |
| PROPYLENE | | | | |
| CARBONATE | | | | |
| ISODODECANE | — | — | — | 1.74 |
| SILICA SILYLATE | — | — | 1.74 | — |
| PERLITE | — | 1.74 | — | — |
| Porous Alumina Particles | 1.74 | — | — | — |
| Acrylates/ | 8.70 | 8.70 | 8.70 | 8.70 |
| Polytrimethyl- | | | | |
| siloxymethacrylate | | | | |
| Copolymer | | | | |
| (40% active content | | | | |
| in Isododecane) | | | | |
| Hydrophobically modified | 13.00 | 13.00 | 13.00 | 13.00 |
| Pigments | | | | |

### Example 2—Rheology

Compositions prepared above were tested on a TA Instruments DHR-3 Rheometer by running an amplitude sweep at 1 rad/s from 0.01% to 100% amplitude. Modulus values were obtained from the resulting curves in the Linear Viscoelastic Region. G′ values at ˜0.1% oscillation strain and are reported below (which fell within the Linear Viscoelastic Region for all samples). It was observed that the G′ values for the porous alumina particles in Invention Composition A had substantially lower modulus as compared to comparative compositions containing other fillers (comparative compositions B and C) as well as lower modulus as compared to even comparative composition D which contained no filler.

| | | Storage |
|---|---|---|
| Formula | Composition | modulus (Pa) |
| Porous Alumina | A | 40.60 |
| Perlite | B | 85.06 |
| Silica Silylate | C | 436.62 |
| No Filler | D | 56.92 |

### Example 3—Pullulan Film Former

Compositions were prepared as in Example 1 above, except pullulan film former was added to the compositions. The compositions were tested as in Example 2 above. It was observed that the G′ values for the porous alumina particles in Invention Composition E had lower modulus as compared to comparative compositions containing other fillers (comparative compositions B and C).

| | Invention E | Comparative F | Comparative G | Comparative H |
|---|---|---|---|---|
| | (Porous Alumina) | (Perlite) | (Silica Silylate) | (No Filler) |
| INCI US NAME | | | | |
| WATER | 32.02 | 32.02 | 32.02 | 32.02 |
| BUTYLENE GLYCOL | 2.61 | 2.61 | 2.61 | 2.61 |
| MAGNESIUM SULFATE | 0.70 | 0.70 | 0.70 | 0.70 |
| CELLULOSE GUM | 0.09 | 0.09 | 0.09 | 0.09 |
| PHENOXYETHANOL | 0.26 | 0.26 | 0.26 | 0.26 |
| ALCOHOL DENAT. | 4.35 | 4.35 | 4.35 | 4.35 |
| POLYGLYCERYL-4 | 2.61 | 2.61 | 2.61 | 2.61 |
| DIISOSTEARATE/ | | | | |
| POLYHYDROXY- | | | | |
| STEARATE/ | | | | |
| SEBACATE | | | | |
| ISOHEXADECANE | 8.70 | 8.70 | 8.70 | 8.70 |
| ISODODECANE | 9.28 | 9.28 | 9.28 | 9.28 |
| DISTEARDIMONIUM | 13.05 | 13.05 | 13.05 | 13.05 |
| HECTORITE (and) | | | | |
| PROPYLENE | | | | |
| CARBONATE | | | | |
| ISODODECANE | — | — | — | 1.74 |
| SILICA SILYLATE | — | — | 1.74 | — |
| PERLITE | — | 1.74 | — | — |
| Porous Alumina Particles | 1.74 | — | — | — |
| TRIMETHYL- | 11.60 | 11.60 | 11.60 | 11.60 |
| SILOXYSILYL | | | | |
| CARBAMOYL | | | | |
| PULLULAN | | | | |
| (30% Active content | | | | |
| in Isododecane) | | | | |
| Hydrophobically modified | 13.00 | 13.00 | 13.00 | 13.00 |
| Pigments | | | | |

| | | Storage |
|---|---|---|
| Formula | Composition | modulus (Pa) |
| Porous Alumina | E | 268.440 |
| Perlite | F | 317.180 |
| Silica Silylate | G | 1086.66 |
| No Filler | H | 252.159 |

### Example 4—Comparative (MQ Resin Film Former)

Compositions were prepared as in Example 1 above, except MQ resin film former was added to the compositions. The compositions were tested as in Example 2 above. It was observed that the G′ values for the porous alumina particles in Composition J does not have the lowest modulus of the comparative compositions containing MQ resin.

| | J (Porous | K | L (Silica | M |
|---|---|---|---|---|
| | Alumina) | (Perlite) | Silylate) | (No Filler) |
| INCI US NAME | | | | |
| WATER | 32.02 | 32.02 | 32.02 | 32.02 |
| BUTYLENE GLYCOL | 2.61 | 2.61 | 2.61 | 2.61 |
| MAGNESIUM SULFATE | 0.70 | 0.70 | 0.70 | 0.70 |
| CELLULOSE GUM | 0.09 | 0.09 | 0.09 | 0.09 |
| PHENOXYETHANOL | 0.26 | 0.26 | 0.26 | 0.26 |
| ALCOHOL DENAT. | 4.35 | 4.35 | 4.35 | 4.35 |
| POLYGLYCERYL-4 | 2.61 | 2.61 | 2.61 | 2.61 |
| DIISOSTEARATE/ | | | | |
| POLYHYDROXY- | | | | |
| STEARATE/ | | | | |
| SEBACATE | | | | |
| ISOHEXADECANE | 8.70 | 8.70 | 8.70 | 8.70 |
| ISODODECANE | 16.24 | 16.24 | 16.24 | 16.24 |
| DISTEARDIMONIUM | 13.05 | 13.05 | 13.05 | 13.05 |
| HECTORITE (and) | | | | |
| PROPYLENE | | | | |
| CARBONATE | | | | |
| ISODODECANE | — | — | — | 1.74 |
| SILICA SILYLATE | — | — | 1.74 | — |
| PERLITE | — | 1.74 | — | — |
| Porous Alumina Particles | 1.74 | — | — | — |
| TRIMETHYL- | 4.64 | 4.64 | 4.64 | 4.64 |
| SILOXYSILICATE | | | | |
| (75% Active in | | | | |
| Isododecane) | | | | |
| Hydrophobically modified | 13.00 | 13.00 | 13.00 | 13.00 |
| Pigments | | | | |

| | | Storage |
|---|---|---|
| Formula | Composition | modulus (Pa) |
| Porous Alumina | J | 149.966 |
| Perlite | K | 138.398 |
| Silica Silylate | L | 512.769 |
| No Filler | M | 116.890 |

What is claimed is:

1. A cosmetic composition comprising, in a physiologically acceptable medium, flattened, porous ceramic particles and at least one film forming agent comprising a hydrocarbon-based backbone and silicone attachment(s) or sidechain(s).

2. The composition of claim 1, in the form of a water-in-oil emulsion.

3. The composition of claim 1, wherein the at least one film forming agent is selected from the group consisting of acrylates/polytrimethylsiloxymethacrylate copolymer, trimethylsiloxysilylcarbamoyl pullulan, and mixtures thereof.

4. The composition of claim 1, further comprising at least one compound selected from the group consisting of at least one thickening agent, at least one colorant, and at least one active agent.

5. The composition of claim 1, wherein the composition is free of thickening agents.

6. The composition of claim 1, wherein the composition is free of coloring agents.

7. The composition of claim 4, wherein at least one coloring agent is present in the composition, and the at least one coloring agent is a surface-treated pigment.

8. The composition of claim 4, wherein at least one active agent is present in the composition, and the at least one active agent is a vitamin, preferably a vitamin B compound.

9. The composition of claim 1, wherein the flattened, porous ceramic particles are alumina particles.

10. The composition of claim 9, wherein the alumina particles have a porosity of from about 5% to about 60%, preferably from about 10% to about 50%.

11. The composition of claim 9, wherein the pores in the alumina particles have of a size of from about 50 nm to about 1000 nm, as measured by Mercury intrusion porosimetry (MIP).

12. The composition of claim 9, wherein the diameter (d) of the alumina particles is greater than 15 microns (μm), preferably greater than 20 microns.

13. The composition of claim 9, wherein the alumina particles have a flattening property (f) of at least 0.3.

14. The composition of claim 1, wherein the composition has a storage modulus (G′) of less than 300 Pa at about 0.1% strain.

15. A method of making up skin comprising applying the composition of claim 1 to skin in an amount sufficient to make up the skin.
